(19) United States
(12) Patent Application Publication
(10) Pub. No.: US 2022/0096561 A1
(43) Pub. Date: Mar. 31, 2022

(54) INHIBITING OR ALLEVIATING AGENT FOR INFLAMMATION IN THE BRAIN

(71) Applicants: Jun LIU, Guangzhou, Guangdong (CN); NIPPON ZOKI PHARMACEUTICAL CO., LTD., Osaka-shi, Osaka (JP)

(72) Inventors: Jun LIU, Guangzhou (CN); Wang LIAO, Guangzhou (CN); Wenli FANG, Guangzhou (CN); Shengnuo FAN, Guangzhou (CN); Yuqiu ZHENG, Shenzhen (CN)

(73) Assignees: Jun LIU, Guangzhou, Guangdong (CN); NIPPON ZOKI PHARMACEUTICAL CO., LTD., Osaka-shi, Osaka (JP)

(21) Appl. No.: 17/426,349

(22) PCT Filed: Jan. 30, 2019
(86) PCT No.: PCT/CN2019/073846

§ 371 Date: Jul. 28, 2021

(51) Int. Cl. A61K 35/30; C12N 5/079

(52) U.S. Cl. CPC A61K 35/30; C12N 2502/70; C12N 5/0622

(57) ABSTRACT

An inhibiting or alleviating agent for inflammation in the brain comprising an extract from inflamed tissue inoculated with vaccinia virus as the active ingredient. In another aspect, the invention relates to a determination or evaluation method of an extract from inflamed tissue inoculated with vaccinia virus or an agent comprising the extract, characterized in that the inhibition of the expression of pro-inflammatory cytokines and/or NF-κB pathway related proteins induced by the promotion of expression of BDNF in cultivated glial cells is used as an indicator. In still another aspect, the invention also relates to a use of an extract from inflamed tissue inoculated with vaccinia virus in the production of the inhibiting or alleviating agent for inflammation in the brain.

### FIELD OF THE INVENTION

The present invention relates to an inhibiting or alleviating agent for inflammation in the brain including an extract from inflamed tissues inoculated with vaccinia virus (hereinafter, it may be mentioned as “the extract”).

### BACKGROUND OF THE INVENTION

Alzheimer's disease (AD) is the most prevalent cause of dementia, which affects 1 in 10 people over 65 years of age. AD characteristically causes extracellular accumulation of amyloid β (Aβ), which forms plaques. There is convincing evidence that Aβ binds to inflammatory receptors (such as TNFR1 and IL-1R) and activates inflammation during AD. Immune-related receptors play an important role on learning and memory formation and excessive neuroinflammation can result in direct cognition impairment. Importantly, synaptic pruning can be regulated by inflammatory signals and chronic neuroinflammation can lead to synaptic-associated proteins loss. Also, it was reported that microglia caused synaptic pruning dysfunction and synaptic loss.

As projected lifespans increase worldwide, treating AD has become an urgent international health priority. The two main AD drugs currently available are donepezil, a cholinesterase inhibitor that increases synaptic acetylcholine (Ach) to enhance cognition in mild AD, and memantine, an N-methyl-D-aspartate receptor (NMDAR) antagonist that reduces excitotoxic neuroinflammation in severe AD. Neither drug stops the progressive cognitive decline, and since 2003 no new drugs have been approved by the Food and Drug Administration (FDA).

Neurotropin (trademark; product of Nippon Zoki Pharmaceutical Co., Ltd.) (hereinafter mentioned as “NTP”) is a well-known analgesic derived from inflamed rabbit skin inoculated with vaccinia virus. For the past 50 years, NTP has been prescribed for neuropathic pain, and its safety is well-established. More recent animal experiments suggest NTP (Most experiments were conducted using experimental product containing the extract in higher concentration than commercial product “Neurotropin”. However the word “the extract” is also used in such cases for convenience sake in this application.) may have significant neuroprotective effects as well. Three months of NTP treatment rescued the spatial cognitive impairment of Ts65Dn mice, a Downs Syndrome model with triplication of 65% of human trisomy-21 genes. NTP treatment also reduced the volume of infarcted lesions, brain edema, and the resulting neurological deficits, and enhanced spatial learning in C57BL/6J mice. Our recent work showed that NTP could alleviate oxidative stress in APP/PS1 mice, an AD model (See Non-Patent Document 1), and inhibits neuroinflammation in BV-2 cells (See Non-Patent Document 2). However, NTP's treatment potential in memory impairment and neuroinflammation during AD has not yet been evaluated.

BDNF plays a pivotal role in modulation of synaptic plasticity, neuronal maintenance, cell survival, neurotransmitter and neurogenesis, and thus in the maintenance of learning and memory. Patients with Alzheimer's disease often have reduced BDNF concentration in their blood and cerebrospinal fluid. Evidence showed that the analgesic effect of NTP probably involved the descending pain inhibitory system via the induction of BDNF. Also, growing evidence has shown that BDNF has modulatory functions on neuroinflammation. NF-κB is a ubiquitous transcriptional factor and it can modulate the expression of inflammatory molecules by translocating into the nucleus and triggering transcription of target genes. There is evidence that responsive sites for immune-related transcriptional factors including NF-kB are in the regulatory promoter region of the genes controlling the expression of APP. In a mice experiment, genetic knockout of the TNF receptor reduces β-secretase1(BACE1) expression which is mediated by NF-kB. Interestingly, this process is also associated with reduced Aβ and enhanced cognitive function.

This study evaluates NTP's effects on cognitive dysfunction and neuroinflammation in an AD transgenic mouse model, and examines molecular mechanisms involved.

### PRIOR ART DOCUMENTS

### Non-Patent Documents

1. Fang W L, Zhao D Q, Wang F, Li M, Fan S N, Liao W, Zheng Y Q, Liao S W, Xiao S H, Luan P and Liu J. Neurotropin (R) alleviates hippocampal neuron damage through a HIF-1/MAPK pathway. Cns Neuroscience & Therapeutics 2017; 23: 428-437.
2. Zheng Y, Fang W, Fan S, Liao W, Xiong Y, Liao S, Li Y, Xiao S and Liu J. Neurotropin inhibits neuroinflammation via suppressing NF-kappaB and MAPKs signaling pathways in lipopolysaccharide-stimulated BV2 cells. J Pharmacol Sci 2018; 136: 242-248.

### SUMMARY OF THE INVENTION

In one aspect, the invention relates to an inhibiting or alleviating agent for inflammation in the brain comprising an extract from inflamed tissue inoculated with vaccinia virus as the active ingredient.

In a preferred embodiment, the inhibition or alleviation of inflammation in the brain is induced by the promotion of intracellular signaling via BDNF-TrkB.

In another preferred embodiment, the activation of glial cells is inhibited by the promotion of intracellular signaling.

In still another preferred embodiment, the glial cells are microglia or astrocytes.

In a further embodiment, the activation of NF-κB pathway related protein is inhibited by the promotion of intracellular signaling.

In a still further embodiment, the NF-κB pathway related protein is IκB or p65.

In a still further embodiment, the inhibition or alleviation of inflammation in the brain is induced by the inhibition of the expression of pro-inflammatory cytokine.

In a still further embodiment, the pro-inflammatory cytokine is 1L-1β, IL-6 or TNF-α.

In a still further embodiment, the agent is for prevention, alleviation, progression control or treatment of Alzheimer's disease.

In a still further embodiment, the inflamed tissue is the skin tissue of rabbits.

In a still further embodiment, the agent is an injection agent or an oral agent.

In another aspect, the invention also relates to a determination or evaluation method of an extract from inflamed tissue inoculated with vaccinia virus or an agent comprising the extract, characterized in that the inhibition of the expression of pro-inflammatory cytokines and/or NF-κB pathway related proteins induced by the promotion of expression of BDNF in cultivated glial cells is used as an indicator.

In a preferred embodiment, the cultivated glial cells are BV-2 cells.

In another preferred embodiment, the pro-inflammatory cytokine is 1L-1β, IL-6 or TNF-α.

In still another preferred embodiment, the NF-κB pathway related protein is IκB or p65.

In a further embodiment, the inflamed tissue is the skin tissue of rabbits.

In still another aspect, the invention also relates to a use of an extract from inflamed tissue inoculated with vaccinia virus in the production of the inhibiting or alleviating agent for inflammation in the brain.

In a preferred embodiment, the inhibition or alleviation of inflammation in the brain is induced by the promotion of intracellular signaling via BDNF-TrkB.

### BRIEF DESCRIPTION OF THE DRAWINGS

FIG. 1: A. The escape latencies of the mice in each group of mice. B. The normalized escape latencies of each group of mice. C. Representative path images of the mice finding the platform. D. The average distances of the mice swimming to find the platform. E. The times of the mice swimming across the target quadrants. The results are presented as mean±SE from at least eight mice in each group. **P<0.01, and NS, nonsignificant.

FIG. 2: A. Aβ plaques were detected by Bielschowsky silver staining in the cortex and hippocampus. B. Aβ plaques were detected with immunofluorescent staining in the cortex and hippocampus. C. Quantification of Aβ plaque load using Bielschowsky silver staining. D. Statistical analysis of Aβ plaque burden with immunofluorescent staining. E and G. Soluble and insoluble Aβ₁₋₄₀ in the brain of TG and TG+NTP mice. F and H. Soluble and insoluble Aβ₁₋₄₂ in the brain of TG mice and TG+NTP mice. The results presented as means±SE from six independent experiments. *P<0.05 and **P<0.01 versus TG mice.

FIG. 3: The coronal sections of the cortex and hippocampus in TG group and TG+NTP group of the mice were stained for A. Aβ, Iba1 and DAPI, B. Aβ, GFAP and DAPI. The percentage of the areas of microglial C. and astrocytes D in the cortex and hippocampus. Analysis of the levels of IL-1β (E), IL-6 (F), and TNF (G) in the cortex and hippocampus of each group by ELISA. Data are presented as mean±SE from six mice in each group. *P<0.05, and **P<0.01.

FIG. 4: BDNF was detected with immunofluorescent staining in the cortex (A) and the hippocampus (B) of each group. Analysis of the levels of BDNF (C), NGF (D), and NT-3(E) in the cortex and the hippocampus with ELISA. Data are presented as mean±SE from six mice in each group. *P<0.05, **P<0.01, and N.S., nonsignificant.

FIG. 5: A. Western blot analyses of the levels of p-65 and p-IκB. B. The relative levels of p-P65, p-IκB-α, and β-actin as a loading control in each group of mice, were quantified. Data are presented as mean±SE from at least three mice in each group. *P<0.05, and **P<0.01.

FIG. 6: A-C. IL-1β, IL-6 and TNF-α were found highly expressed after LPS treatment by comparing with control group. IL-1β, IL-6 and TNF-α increased after a selective, non-competitive BDNF receptor antagonist, ANA12, administration. D. Cell viability was assayed by CCK8 after treatment with ANA12. E. BDNF level was detected after NTP and ANA12 treatment. F-H. Both p-p65 and p-IκB-α were activated by LPS and inactivated by NTP. The activation of p-p65 and p-IκB-α was abolished by ANA12.

### MODE FOR CARRYING OUT THE INVENTION

### Materials

As to basic extracting steps for the extract, the following steps are used for example.

(A) Inflamed skin tissues of rabbits, mice etc. by the intradermal inoculation with vaccinia virus are collected, and the inflamed tissues are crushed. To the crushed tissues an extraction solvent such as water, phenol water, physiological saline or phenol-added glycerin water is added to conduct an extracting treatment for several days. Then, the mixture is filtrated or centrifuged to give a crude extract (filtrate or supernatant) wherefrom tissue fragments are removed.

(B) The crude extract obtained in (A) is adjusted to acidic pH, heated and then filtered or centrifuged to conduct a deproteinizing treatment. After that, the deproteinized solution is adjusted to basic pH, heated and then filtered or centrifuged to give a deproteinized filtrate or supernatant.

(C) The filtrate or the supernatant obtained in (B) is adjusted to acidic pH and adsorbed with an adsorbent such as activated carbon or kaolin.

(D) An extraction solvent such as water is added to the adsorbent obtained in (C), the mixture is adjusted to basic pH and the adsorbed component is eluted to give an extract from inflamed skins of rabbits inoculated with vaccinia virus (the present extract).

Various animals which can be infected with vaccinia virus such as rabbit, bovine, horse, sheep, goat, monkey, rat, mouse, etc. can be used as an animal for vaccinating vaccinia virus and obtaining inflamed tissue. Among them, an inflamed skin tissue of a rabbit is preferable as an inflamed tissue. Any rabbit may be used so far as it belongs to Lagomorpha. Examples thereof include Oryctolagus cuniculus, domestic rabbit (domesticated Oryctolagus cuniculus), hare (Japanese hare), mouse hare and snowshoe hare. Among them, it is appropriate to use domestic rabbit. In Japan, there is family rabbit called “Kato” which has been bred since old time and frequently used as livestock or experimental animal and it is another name of domestic rabbit. There are many breeds in domestic rabbit and the breeds being called Japanese white and New Zealand white are advantageously used.

Vaccinia virus used herein may be in any strain. Examples thereof include Lister strain, Dairen strain, Ikeda strain, EM-63 strain and New York City Board of Health strain.

More detailed description regarding the method of manufacturing the extract is described, for example, in the paragraphs [0024]′ [0027], [0031], etc. of WO2016/194816.

Aβ₂₅₋₃₅ was synthesized by Shanghai Sangon Biological Engineering Technology & Services Co. (Shanghai, China). Fetal bovine serum (FBS), medium (DMEM), neurobasal medium, and N2 supplement were obtained from Gibco (New York, USA). A cell counting kit-8 (CCK-8) was acquired from Dojin Kagaku (Kumamoto, Kyushu, Japan). Apoptosis detection kit was purchased from eBioscience (San Diego, Calif., USA). A ROS detection kit and mitochondrial membrane potential assay kit with JC-1 were purchased from the Beyotime Institute of Biotechnology (Shanghai, China). Hoechst 33342 and propidium iodide (PI) were procured from Invitrogen/Life Technologies (Carlsbad, Calif., USA). SOD, GSH, MDA, and CAT kits were supplied by Jiancheng Bioengineering Institute (Nanjing, China). The following primary antibodies against p-Erk1/2, p-P38, p-JNK, Erk1/2, P38, JNK, Bcl-2, Bax and secondary antibody horseradish peroxidase-(HRP−) conjugated goat anti-rabbit IgG were obtained from Cell Signaling Technology (Danvers, Mass., USA). The primary antibody against HIF-1α was obtained from Abcam (Cambridge, Mass., USA) and the primary antibody against Aβ₁₋₄₂ was purchased from Sigma-Aldrich (St. Louis, Mo., USA). The chemiluminescent horseradish peroxidase substrate was purchased from Millipore (Billerica, Mass., USA). All other routine experimental supplies and reagents were acquired from Thermo Fisher, Invitrogen, and MR Biotech.

### EXAMPLES

As to basic extracting steps for the extract, the following steps are used for example.

(A) Inflamed skin tissues of rabbits, mice etc. by the intradermal inoculation with vaccinia virus are collected, and the inflamed tissues are crushed. To the crushed tissues an extraction solvent such as water, phenol water, physiological saline or phenol-added glycerin water is added to conduct an extracting treatment for several days. Then, the mixture is filtrated or centrifuged to give a crude extract (filtrate or supernatant) wherefrom tissue fragments are removed.

(B) The crude extract obtained in (A) is adjusted to acidic pH, heated and then filtered or centrifuged to conduct a deproteinizing treatment. After that, the deproteinized solution is adjusted to basic pH, heated and then filtered or centrifuged to give a deproteinized filtrate or supernatant.

(C) The filtrate or the supernatant obtained in (B) is adjusted to acidic pH and adsorbed with an adsorbent such as activated carbon or kaolin.

(D) An extraction solvent such as water is added to the adsorbent obtained in (C), the mixture is adjusted to basic pH and the adsorbed component is eluted to give an extract from inflamed skins of rabbits inoculated with vaccinia virus (the present extract).

Various animals which can be infected with vaccinia virus such as rabbit, bovine, horse, sheep, goat, monkey, rat, mouse, etc. can be used as an animal for vaccinating vaccinia virus and obtaining inflamed tissue. Among them, an inflamed skin tissue of a rabbit is preferable as an inflamed tissue. Any rabbit may be used so far as it belongs to Lagomorpha. Examples thereof include Oryctolagus cuniculus, domestic rabbit (domesticated Oryctolagus cuniculus), hare (Japanese hare), mouse hare and snowshoe hare. Among them, it is appropriate to use domestic rabbit. In Japan, there is family rabbit called “Kato” which has been bred since old time and frequently used as livestock or experimental animal and it is another name of domestic rabbit. There are many breeds in domestic rabbit and the breeds being called Japanese white and New Zealand white are advantageously used.

Vaccinia virus used herein may be in any strain. Examples thereof include Lister strain, Dairen strain, Ikeda strain, EM-63 strain and New York City Board of Health strain.

More detailed description regarding the method of manufacturing the extract is described, for example, in the paragraphs [0024]′ [0027], [0031], etc. of WO2016/194816.

### (1) Mice and Drug Administration

APPswe/PS1dE9 (APP/PS1) double transgenic mice were purchased from the Model Animal Research Center of Nanjing University (Nanjing, China). These mice model AD through the chimeric insertion of human amyloid precursor protein (APP) and human presenilin1 (PS1) genes, which are overexpressed in patients with early-onset AD. 24 6-month-old APP/PS1 males and 24 wild-type litter-mate controls were housed in specific pathogen free (SPF) conditions on a 12 h light/dark cycle with free access to food and water, and all were handled according to the protocols of the Institutional Animal Care and Use Committee of Sun Yat-sen University, Guangzhou, China. Half of the mice from each genotype were randomly chosen to receive 200 NU/kg NTP or 0.9% NaCl placebo, given by daily oral gavage for three months (n=12 in each group). After treatment, when they were 9 months old, the mice were behaviorally tested and then sacrificed to analyse biochemically.

### (2) Cell Culture

Immortal BV-2 murine microglial cells, a gift from Dr. Ying Chen of Sun Yat-sen Memorial Hospital, Sun Yat-sen University were cultured as described (refer Non-Patent Document 2). BV-2 cultures were treated with 0.1 NU/mL NTP, then given lipopolysaccharides (1000 ng/mL, LotL2880, O55:B5, Sigma-Aldrich, St. Louis, Mo., USA) 12 h later. Some cultures were pre-treated with 10 uM of selective non-competitive BDNF receptor agonist ANA-12 (Sigma-Aldrich) 1 h before NTP, to demonstrate NTP's action through BDNF pathways (refer Fan D, Li J, Zheng B, Hua L and Zuo Z. Enriched Environment Attenuates Surgery-Induced Impairment of Learning, Memory, and Neurogenesis Possibly by Preserving BDNF Expression. Mol Neurobiol 2016; 53: 344-354. and Liu S, Li X, Gao J, Liu Y, Shi J and Gong Q. Icariside II, a Phosphodiesterase-5 Inhibitor, Attenuates Beta-Amyloid-Induced Cognitive Deficits via BDNF/TrkB/CREB Signaling. Cell Physiol Biochem 2018; 49: 985.).

### (3) Morris Water Maze (MWM)

After three months of NTP or vehicle treatment, the mice were tested for spatial learning and memory in the Morris water maze as previously described (refer Xiao S H, Zhou D Y, Luan P, Gu B B, Feng L B, Fan S N, Liao W, Fang W L, Yang L H, Tao E X, Guo R and Liu J. Graphene quantum dots conjugated neuroprotective peptide improve learning and memory capability. Biomaterials 2016; 106: 98-110.). Briefly, they were given four consecutive trials per day, starting in a different quadrant for each trial. Trials lasted 90 seconds and ended when the mice successfully reached the platform and stayed there for 5 s. If mice could not find the platform in 90 s, the experimenter manually set them there and let them stay for 20 s.

Each mouse's time to find the platform on the first day was normalized at 1, then used to normalize the and platform times on subsequent days were normalized to the previous day (latency day n/latency day n−1), to calculate a learning trend. The relative escape latencies in the following training day to that of the first day were analyzed (escape latency in the following day/escape latency in the first day) and labeled as learning trend. The probe trial was conducted 24 h after the end of the acquisition trial when the platform was removed. In our experiment, the latency to the primary target site, the time spent in the target quadrant, and the numbers of platform-site crossovers within 60 s were recorded.

### (4) Bielschowsky Silver Staining and Immunofluorescent Staining

Bielschowsky silver staining and immunofluorescent staining were performed on fixed sections as described previously (refer Knezovic A, Osmanovic-Barilar J, Curlin M, Hof P R, Simic G, Riederer P and Salkovic-Petrisic M. Staging of cognitive deficits and neuropathological and ultrastructural changes in streptozotocin-induced rat model of Alzheimer's disease. J Neural Transm (Vienna) 2015; 122: 577-592. and Liu J, Rasul I, Sun Y, Wu G, Li L, Premont R T and Suo W Z. GRKS deficiency leads to reduced hippocampal acetylcholine level via impaired presynaptic M2/M4 autoreceptor desensitization. J Biol Chem 2009; 284: 19564-19571.). Bielschowsky silver staining was used to assess Aβ and immunofluorescence was used to evaluate levels of Aβ deposits, BDNF expression, and the area of GFAP⁺ and Iba1⁺ cells in the hippocampus and cortex of each group. The primary antibodies used in immunofluorescent staining were as following: rabbit anti-Aβ (1:100, Abcam, MA, USA), rabbit anti-BDNF (1:500; Millipore, Mass., USA), goat anti-GFAP (1:1000; Abcam, MA, USA), goat anti-Iba1 (1:500; Abcam, MA, USA). DAPI (Invitrogen, CA, USA) was used to detect nuclei. Images were acquired from a fluorescent microscope. The area of Aβ plaques, GFAP⁺ cells, and Iba1⁺ cells in the cortex and hippocampus in each image were quantified by Image J (National Institutes of Health, Md., USA).

### (5) Enzyme-Linked Immunosorbent Assay (ELISA)

The brain samples (separated into the cortex and the hippocampus) were stored at −80° C. till analysis. We measured the concentration of Aβ₁₋₄₀, Aβ₁₋₄₂, BDNF, NGF, NT-3, IL-1β, IL-6 and TNF-α with the ELISA method at 9 months of age, which have been administrated with NTP for 3 months (refer Non-Patent Document 2). The assays were performed using commercially available ELISA kits (Invitrogen for Aβ₁₋₄₀, Aβ₁₋₄₂, IL-6, IL-1β and TNF-α, Promega for BDNF, and CUSABIO for NGF and NT-3) according to the manufacturer's instructions. The total protein concentration was determined using the BCA Protein Assay kit (Thermo Scientific, USA). Absorbance of the samples was detected with a multifunctional microplate reader (SpectraMax M5, Sunnyvale, Calif., USA).

### (6) Western Blot Analysis

Western blotting and semi-quantitative analyses were performed following previously described procedures (refer Liao W, Jiang M J, Li M, Jin C L, Xiao S H, Fan S N, Fang W L, Zheng Y Q and Liu J. Magnesium Elevation Promotes Neuronal Differentiation While Suppressing Glial Differentiation of Primary Cultured Adult Mouse Neural Progenitor Cells through ERK/CREB Activation. Frontiers in Neuroscience 2017; 11:). In brief, proteins in cerebral cortex and hippocampus were extracted with lysis buffer for 30 min, followed by centrifugation at 14,000 rpm for 15 min at 4.0 to obtain the supernatant for western blot analysis. Primary antibodies and dilution rates used were listed as follow: NF-κB (p65), 1:1000; p-IκBα, 1:500 and β-actin, 1:1000. Primary antibodies against NF-κB (p65), p-IκBα and β-actin were purchased from Cell Signaling Technology Inc (MA, USA). Horseradish peroxidase-conjugated secondary antibodies were used, and the bands were fixed and visualized by an ECL advanced kit. β-actin was utilized as an internal control for protein loading and transfer efficiency. Western blot assay results reported here are representative of at least 3 experiments. The quantification of protein expression was analyzed by Image J (National Institutes of Health, Md., USA).

### (7) CCK-8 Assay for Cell Viability

The effects of ANA-12 on BV-2 cells viability were detected by CCK-8 assay (refer Fan D, Li J, Zheng B, Hua L and Zuo Z. Enriched Environment Attenuates Surgery-Induced Impairment of Learning, Memory, and Neurogenesis Possibly by Preserving BDNF Expression. Mol Neurobiol 2016; 53: 344-354.). In brief, cells were cultured on a 96-well plate at a density of 1×10⁴ per well for 24 h and then administrated with ANA12 (5 uM, 10 uM, 15 uM) for another 24 h. Then the cells were incubated at 37 C for 2 h and the absorbance values of the samples were measured at 450 nm by a multifunctional microplate reader (SpectraMax M5, Sunnyvale, Calif., USA).

### (8) Statistical Analysis

SPSS 16.0 for Windows (SPSS Inc., Chicago, Ill., USA) was used to carry out the statistical analyses. Two-way analysis of variance (ANOVA) with repeated measures was used to analyze the MWM data. Other statistical tests were conducted using one-way ANOVA and Student's t-test for comparisons between groups. The data were expressed as the mean±SE, and differences were considered statistical significance at P<0.05.

### (9) Results

### (i) Chronic NTP Treatment Attenuates Cognitive Deficits of APP/PS1 Mice in the Morris Water Maze

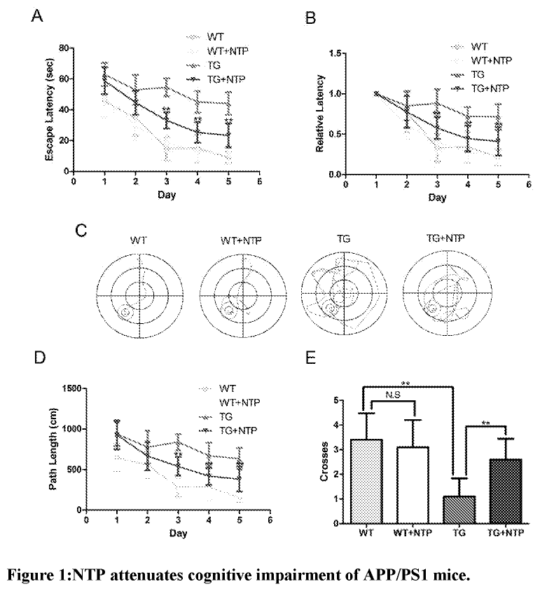

Morris water maze test was performed to evaluate whether NTP could attenuate the cognitive deficits in the APP/PS1 transgenic mice at 9 months of age (FIG. 1). The NTP-treated APP/PS1 mice were administrated with NTP at 6 months of age for 3 months by oral gavage delivery. The control APP/PS1 mice were administrated with saline (0.9% NaCl). During the hidden platform tests, control WT mice showed progressively decreased in the escape latencies over the consecutive 5 days of training. Control APP/PS1 mice had a slight decline in the escape latencies during the entire training periods, but there was a significant extention in escape latency time compared with WT mice (P<0.01, FIG. 1A). To control individual differences in swimming speed, we also normalized the escape latencies of each group in the first trial day to 1.0 (FIG. 1B). Compared with WT mice, control APP/PS1 mice still show a failure in learning trend, indicating impaired learning ability (P<0.01, FIG. 1B). In contrast, NTP-treated APP/PS1 mice exhibited a comparable learning trend with WT mice. Similar to the escape latencies, NTP-treated APP/PS1 mice showed progressively decreased in the swimming length compared with control APP/PS1 mice (P<0.01, FIGS. 1C and D). In the probe test, NTP-treated APP/PS1 mice tended to concentrate in the target area of the pool and cross over the target quadrant more times than control APP/PS1 mice (P<0.01, FIG. 1E). NTP-treated mice were similar to control WT mice and no significant differences were observed in escape latencies, path length, and numbers of platform area crossings. These results demonstrate that chronic NTP treatment can improve cognitive deficits in APP/PS1 mice.

### (ii) Chronic NTP Treatment Reduces Aβ Burden in APP/PS1 Mice

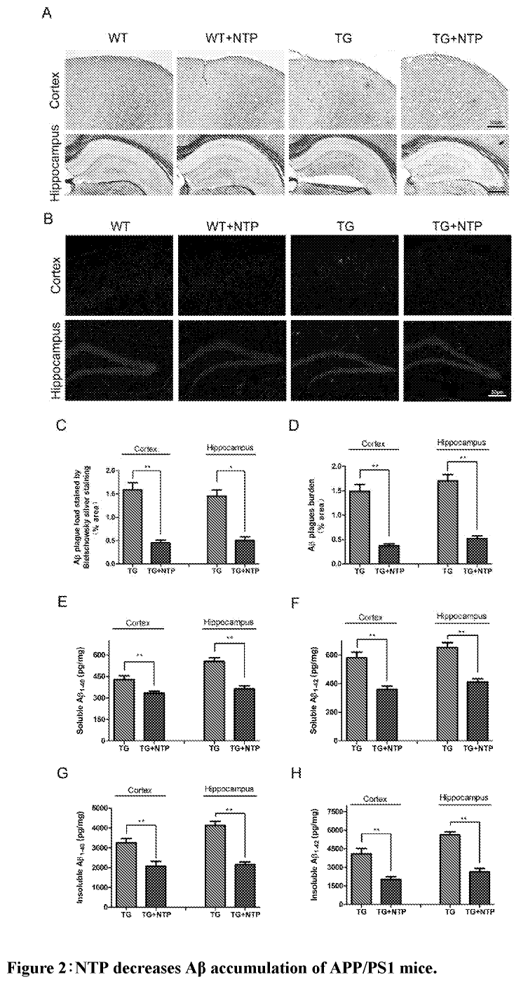

To examine the potential function of NTP treatment on Aβ aggregation and to observe the morphologic changes after NTP treatment, the slices of the cortex and hippocampus of four groups of mice were stained using both Bielschowsky silver staining and immunofluorescent staining (FIGS. 2A and B). Quantification analysis revealed that the APP/PS1 mice treated with NTP showed significantly lower amyloid plaques in both the cortical and hippocampal areas than the control APP/PS1 mice (P<0.01, FIGS. 2C and D). Moreover, previous studies have shown that APP/PS1 mice have age-related increased levels in both soluble and insoluble Aβ₁₋₄₀ and Aβ₁₋₄₂ (refer Zhou J, Ping F F, Lv W T, Feng J Y and Shang J. Interleukin-18 directly protects cortical neurons by activating PI3K/AKT/NF-kappaB/CREB pathways. Cytokine 2014; 69: 29-38. and Grilli M, Ribola M, Alberici A, Valerio A, Memo M and Spano P. Identification and characterization of a kappa B/Rel binding site in the regulatory region of the amyloid precursor protein gene. J Biol Chem 1995; 270: 26774-26777.). Consistent with decreased Aβ burden, ELISA analysis demonstrated that NTP-treated APP/PS1 mice showed a significant decline in both soluble Aβ₁₋₄₀ and Aβ₁₋₄₂ levels compared with that in both the hippocampus and cortex of APP/PS1 mice (P<0.05, FIG. 2E, FIG. 2F). For insoluble Aβ₁₄₀ and Aβ₁₄₂, we also found a significant decrease in NTP-treated APP/PS1 mice (P<0.05, FIG. 2G, FIG. 2H). These results suggest that chronic treatment with NTP may be able to have an inhibitory effect on the generation and accumulation of Aβ plaques in the brain of APP/PS1 mice.

(iii) Chronic NTP Treatment Inhibits Glial Activation in APP/PS1 Mice

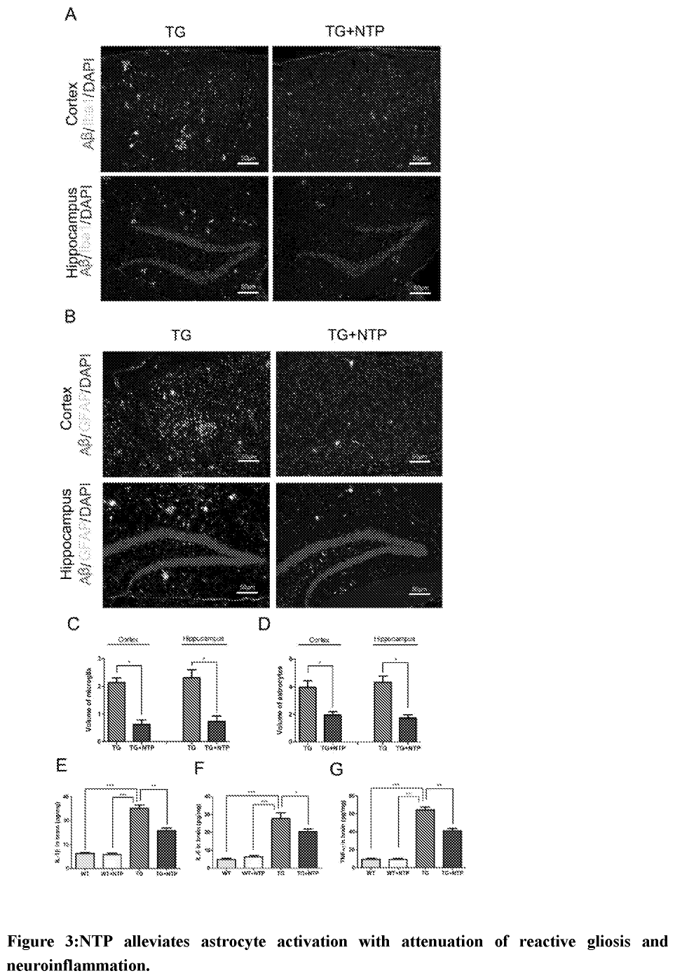

Activated microglia and astrocytes have been shown to be associated with Aβ accumulation, and they can promote the production of pro-inflammatory cytokines, resulting in synaptic dysfunction, neuronal death, and neurodegeneration. Therefore, we examined whether NTP treatment might alter glial activation in the cerebral cortex and hippocampus of APP/PS1 mice at 9 months of age, using immunofluorescent staining with antibodies against ionized calcium-binding adaptor molecule 1 (Iba-1) and glial fibrillary acidic protein (GFAP) to reveal changes in microgliosis and astrogliosis. We found that Aβ plaques were surrounded by Iba-1 immunoreactivity (IR) microglia (FIG. 3A) and GFAP-IR astrocytes (FIG. 3B), indicating both microglial and astrocytic activation in the cortex and the hippocampus of control APP/PS1 mice. In contrast, significant decreases in the area percentage of Iba-1-IR microglia was observed accompanied with reduced Aβ burden in NTP-treated APP/PS1 mice (P<0.01, FIG. 3C). Consistently, the area percentage of GFAP-IR astrocytes also reduced after NTP treatment (P<0.01, FIG. 3 D). These results show that chronic NTP treatment may suppress glial activation in APP/PS1 mice.

### (iv) NTP Treatment Decreases Pro-Inflammatory Cytokines in APP/PS1 Mice

Persistent activated microglia and astrocytes can mediate neuroinflammation via releasing pro-inflammatory cytokines and facilitate Aβ deposition, leading to inflammatory neuronal damage. Furthermore, previous evidence has suggested that NTP was able to suppress inflammatory cytokine expression in hepatocytes. Thus, to explore whether chronic treatment with NTP could affect the production of inflammatory factors in 9-month APP/PS1 mice, we examined the levels of pro-inflammation cytokines including interleukin-1 beta (IL-1β), interleukin-6 (IL-6) and tumor necrosis factor-alpha (TNF-α) using ELISA tests. We observed that APP/PS1 mice had markedly higher levels of IL-1β than NTP-treated APP/PS1 mice (P<0.05, FIG. 3E). After NTP treatment, APP/PS1 mice showed decreased IL-6 level (P<0.05, FIG. 3F). Additionally, the level of TNF-α was lower in NTP-treated group when compared with APP/PS1 mice without NTP treatment (P<0.05, FIG. 3G). There was no difference in levels of IL-1β, IL-6 and TNF-α between WT and NTP-treated WT mice. These results demonstrate that NTP may effectively reduce inflammatory reaction, ameliorating neuroinflammation in APP/PS1 mice.

### (v) NTP Treatment Promotes BDNF Expression in APP/PS1 Mice

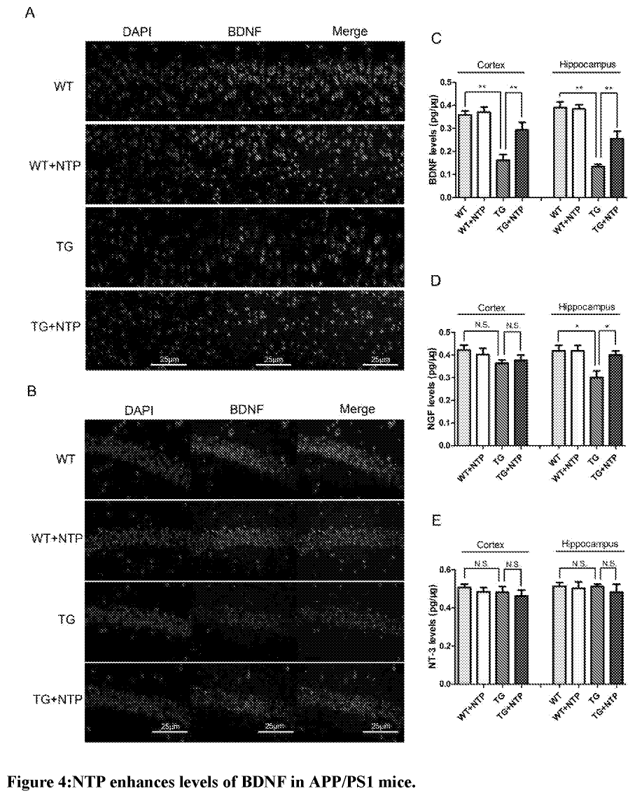

Brain-derived neurotrophic factor (BDNF), a member of the neurotrophin family, is vital for synaptic plasticity and neuronal survival, and it is critical for learning and memory. It has come to light that BDNF was able to attenuate proinflammatory cytokines production, demonstrating that BDNF may be correlated with homeostatic maintenance during neuroinflammation (refer Lima Giacobbo B, Doorduin J, Klein H C, Dierckx R, Bromberg E and de Vries E F J. Brain-Derived Neurotrophic Factor in Brain Disorders: Focus on Neuroinflammation. Mol Neurobiol 2018;). Thus, we further explored the changes of BDNF expression in each group of mice by immunostaining (FIGS. 4A and B) and ELISA (P<0.01, FIG. 4C).

The results showed that 9-month old APP/PS1 mice had significantly lower levels of BDNF in both the cerebral cortex and hippocampus when compared to WT mice. In contrast, BDNF levels were significantly enhanced in the cortex and hippocampus of NTP-treated APP/PS1 mice compared with control APP/PS1 mice (P<0.01, FIG. 4A-C). In addition, upregulated levels of NGF were observed in the hippocampus but not the cortex of NTP-treated APP/PS1 mice compared with control APP/PS1 mice (P<0.05, FIG. 4D). However, we found that levels of neurotrophin-3 (NT-3) remained unchanged among all the groups of mice (P>0.05, FIG. 4E). Together, these findings reveal that chronic NTP treatment could markedly promote the expression of BDNF in the brain of APP/PS1 mice.

### (vi) NTP Regulates NF-κB Pathway In Vivo and In Vitro

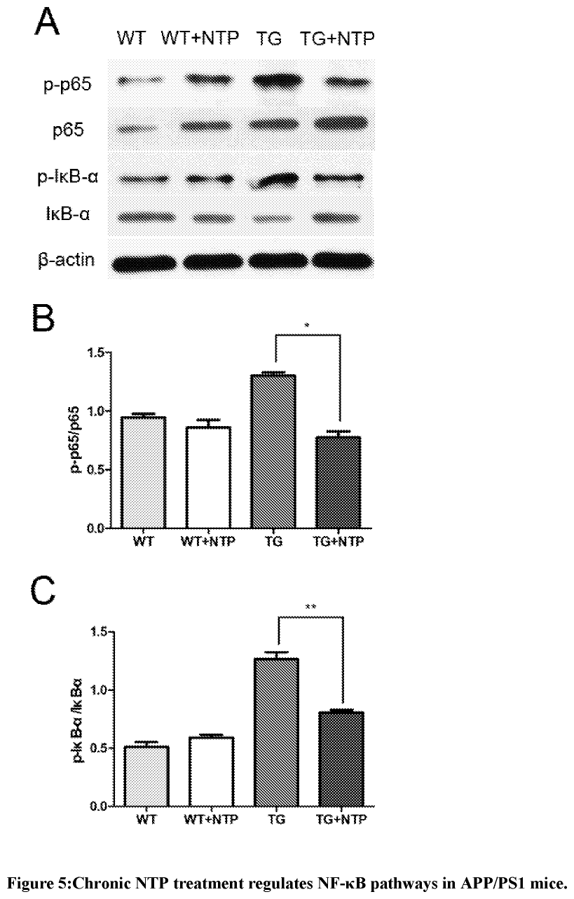

To further explore the underlying mechanism, we assessed the expression of NF-κB pathways related proteins by Western blot analysis. We found that protein expression p-p65 and p-IκB-α were significantly up-regulated in APP/PS1 mice when compared with the WT group. After NTP treatment, p-p65 and p-IκB-α in the APP/PS1 mice were significantly down-regulated (FIG. 5). It suggests that NTP may inhibit neuroinflammation and improve cognitive impairment via BDNF/NF-κB pathway.

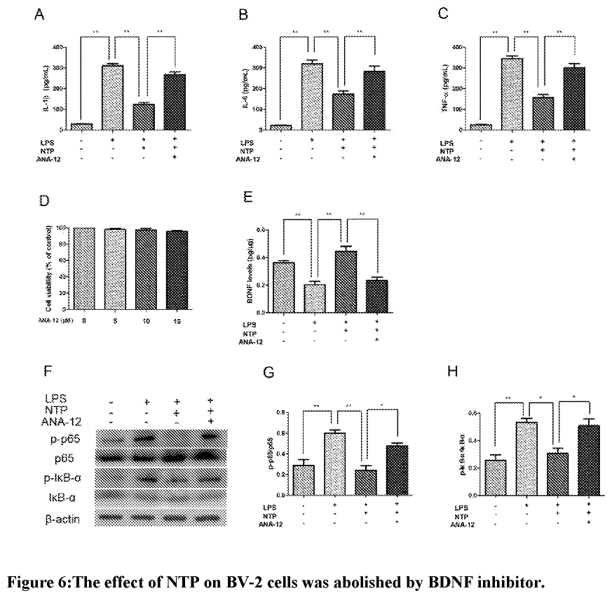

To verify this mechanism, we used LPS to induce inflammation in BV-2 cell. It is shown that IL-1β, IL-6 and TNF-α were found highly expressed after LPS treatment (1000 ng/mL) by comparing with control group (FIG. 6A-C). To further explore the link between BDNF and NF-κB, we used a selective, non-competitive BDNF receptor antagonist, ANA12, to inhibit BDNF pathway. As is shown in the FIG. 6A-C, the expression of IL-1β, IL-6 and TNF-α decreased after NTP treatment but increased after ANA12 administration. Cell viability was assayed by CCK8 after treatment with ANA12 and there was no difference after ANA12 treatment at the concentration of 5 uM, 10 uM and 15 uM (FIG. 6D). Additionally, BDNF level was detected after NTP and ANA12 treatment. LPS reduced BDNF level while NTP increased BDNF level. The effect of NTP on BDNF was abolished by ANA12(FIG. 6E). We also examined the expression of p-p65 and p-IκB-α on LPS-stimulated cells. Consistently, we found that both p-p65 and p-IκB-α were activated by LPS and reduced by NTP. Interestingly, the activation of p-P65 and p-IκB-α were shown to be abolished by ANA-12(FIG. 6F-H). Taken together, our results demonstrated that NTP regulated BDNF/NF-κB pathways in vivo and in vitro.

### INDUSTRIAL APPLICABILITY

NTP is a widely used analgesic drug for the treatment of intractable neuropathic pain. Recently, the potential therapeutic effects of NTP are rapidly expanding. NTP showed capability of protecting the brain against ischemic stroke, accelerates the remyelination in demyelination disease and reduced muscular mechanical hyperalgesia. However, there is still no evidence for the role of NTP play on cognitive function and inflammation in mouse model of AD, which is a multifactorial neurodegenerative disease without effective treatment.

NTP was demonstrated to have function of enhancing spatial learning of C57BL/6J mice. In addition, NTP was found to facilitate cognitive improvement of Ts65Dn mice, a Down Syndrome mouse model. However, there is still no evidence that NTP can have any influence on Alzheimer's disease. Our study shown that chronic NTP treatment was sufficiently to improve cognitive deficits in APP/PS1 mice, which was assessed by Morris water maze test.

Neuroinflammation is a critical feature of AD and activation of microglia and astrocytes by Aβ may promote the production of proinflammatory cytokines, enhancing neuroinflammation reactions. In this study, we chose APP/PS1 mice as our AD transgenic model since this model steadily mimic the behavioral and pathological changes of AD and has been widely used in AD researches. The present study highlighted the inhibition of NTP on neuroinflammation including microgliosis, astrogliosis, and pro-inflammation cytokines (IL-1β, IL-6, and TNF-α) in APP/PS1 mice.

In the present study, we observed that NTP treatment significantly increased the expression of BDNF and inhibitor of BDNF receptor could abolish this effect. It suggests that NTP may play the neuroprotective role in a BDNF dependent manner. Bdnf gene expression has been demonstrated to be regulated by physical activity or pathological stimuli like stress, trauma, infection and aging. BDNF levels are reduced in plasma of patients with AD. Normally, BDNF is translated as pro-neurotrophin (pro-BDNF) that can be cleaved into mature BDNF by endoproteases or metalloproteinases. BDNF can be secreted and bind to the two different kinds of receptors, low affinity p75 neurotrophin receptor (p75NTR) and high-affinity receptor tyrosine kinase B (TrkB). Binding to these two different receptors potentially activates different pathways and leads to either cell death or survival. However, the concentration of pro-BDNF was reported to be ten times lower than mature BDNF in animal model. Therefore, we detected mature BDNF and used the TrkB inhibitor to block the BDNF pathway in the present study.

Microglia participate actively in the development of pathological neuroinflammatory process, which plays an important role in AD pathogenesis. In this study, our results showed that chronic NTP treatment inhibited glial activation and decreased pro-inflammatory cytokines in APP/PS1 mice. In the nervous system, the main factor of neuronal inflammatory activation is NF-kB, a regulator of apoptosis, proliferation, and maturation of immune cells. NF-κB (p65) is bound to IκB as an inactive p65/IκB complex existing in the cytoplasm before its activation. It is reported that activated NF-κB is found surrounding amyloid plaques in AD brain. Frede and colleagues observed that bacterial LPS was able to induce NF-κB up-regulation. In agreement, our recent studies have demonstrated that NTP can suppress the expression of NF-κB in lipopolysaccharide-stimulated BV2 cells. Consistently, present results exhibited that supplementation of NTP markedly decreased the activation of p-p65 and p-IκB-α in APP/PS1 mouse model. However, it is reported that binding of BDNF to the TrkB could also induce the expression of NF-kB. NF-κB stimulated by BDNF might activate PLC-γ/PKC signaling via the kinases IKKα and IKKβ, which subsequently phosphorylates the NF-κB inhibitory unit IκBα. Consequently, binding of ubiquitin and degradation of IκBα by proteasomes induces the release of the NF-κB.

Qirui Bi et al. showed that Venenum bufonis triggers neuroinflammation through NF-κB pathways, leading to an ultimate decrease in BDNF, but they did not directly link NF-κB cytokines with BDNF. Cai et al. report that BDNF protects against IL-1β stimulation by modulating NF-κB signaling. We used a specific BDNF receptor inhibitor, ANA12, to block the BDNF pathway in vitro. This pre-treatment abolished NTP's neuroprotective effects against LPS-stimulated inflammation, supporting the notion that NTP may protect the neuroinflammation via BDNF/NF-κB pathway.

However, the exact mechanism of BDNF functions on NF-κB still remains to be explored. Casein kinase II(CK2) is a highly conserved ubiquitous serine/threonine protein kinase which have been proved to activate NF-κB. It is reported that BDNF upregulated NF-κB by CK2. Also, BDNF was demonstrated to produce neuroprotective effect via ERK1/2 signaling which is consistent with our previous researches. BDNF activate NF-κB via CK2, which seems to be independent of ERK1/2 and PI3K. As is shown by our previous research, NTP cloud decrease the translocation of p65 from cytoplasm to nuclear, which might be a novel mechanism for BDNF to regulate NF-κB activation. Further research needs to be conducted to fully understand the mechanism of BDNF on NF-κB pathway.

These intriguing findings suggest that NTP can counteract neuroinflammation and rescue cognitive deficits of APP/PS1 mice by enhancing through the BDNF/NF-κB pathway. The results provide further insight into the interactions of NTP and neuroinflammation. NTP may be a new promising drug candidate for patients with AD. In addition, although NTP has established safe profiles in humans, it still requires large-scale clinical trials for further confirmation of its neuroprotective capability in both sporadic and familial AD.

1. Aft method for inhibiting or alleviating inflammation in the brain comprising administering an extract from inflamed tissue inoculated with vaccinia virus or an agent comprising the extract to a patient in need thereof.

2. The method according to claim 1, wherein the inhibition or alleviation of inflammation in the brain is induced by the promotion of intracellular signaling via BDNF-TrkB.

3. The method according to claim 2, wherein the activation of glial cells is inhibited by the promotion of intracellular signaling.

4. The method according to claim 3, wherein the glial cells are microglia or astrocytes.

5. The method according to claim 2, wherein the activation of NF-κB pathway related protein is inhibited by the promotion of intracellular signaling.

6. The method according to claim 5, wherein the NF-κB pathway related protein is IκB or p65.

7. The method according to claim 1, wherein the inhibition or alleviation of inflammation in the brain is induced by the inhibition of the expression of pro-inflammatory cytokine.

8. The method according to claim 7, wherein the pro-inflammatory cytokine is 1L-1β, IL-6 or TNF-α.

9. The method according to claim 1, wherein the patient has Alzheimer's disease.

10. The method according to claim 1, wherein the inflamed tissue is the skin tissue of rabbits.

11. The method according to claim 1, wherein the extract or agent is administered by injection.

12. The method according to claim 1, wherein the extract or agent is orally administered.

13. A method comprising contacting cultivated glial cells with an extract from inflamed tissue inoculated with vaccinia virus or an agent comprising the extract, and determining or evaluating whether there is inhibition of the expression of pro-inflammatory cytokine and/or NF-κB pathway related protein induced by the promotion of expression of BDNF in the cultivated glial cells.

14. The method according to claim 13, wherein the cultivated glial cells are BV-2 cells.

15. The method according to claim 13, wherein the pro-inflammatory cytokine is 1L-1β, IL-6 or TNF-α.

16. The method according to claim 13, wherein the NF-κB pathway related protein is IκB or p65.

17. The method according to claim 13, wherein the inflamed tissue is the skin tissue of rabbits.

18. (canceled)

19. (canceled)
